(19) United States
(12) Patent Application Publication
(10) Pub. No.: US 2022/0254518 A1
(43) Pub. Date: Aug. 11, 2022

(54) Patient Information Network

(71) Applicant: Blockchain Applied Technologies, Inc., Sparta, NJ (US)

(72) Inventors: Robert Dufour, Rogers, AR (US); Joseph Macaluso, Rogers, AR (US); Charles St. Charles, Rogers, AR (US)

(21) Appl. No.: 17/669,323

(22) Filed: Feb. 10, 2022

Related U.S. Application Data
(60) Provisional application No. 63/147,852, filed on Feb. 10, 2021.
(60) Provisional application No. 63/308,939, filed on Feb. 10, 2022.

(51) Int. Cl. G16H 80/00; G06F 3/0482; H04L 51/046; G06F 3/0484; G16H 70/40; G16H 20/10

(52) U.S. Cl. CPC G16H 80/00; G06F 3/0482; G16H 20/10; G06F 3/0484; G16H 70/40; H04L 51/046

(57) ABSTRACT

A method and a system are disclosed for direct procurement of drugs by patient consumers from pharmaceutical manufacturers without drug switching by an insurance company. The method is performed on a distributed networking system that includes multiple users on multiple user interfaces that enable the proper identification of symptoms and diseases, the provision of relevant medical solutions and medicine, and financial assistance to the patient consumer to obtain the relevant medicine. Both patient consumer and pharmaceutical manufacturer are advantaged by having a direct drug delivery exchange without the involvement of a non-physician third party.

### CROSS-REFERENCE TO RELATED APPLICATIONS

This application claims priority pursuant to 35 U.S.C. 119(e) to co-pending U.S. Provisional Patent Application Ser. No. 63/147,852 filed Feb. 10, 2021, and U.S. Provisional Patent Application Ser. No. 63/308,939 filed Feb. 10, 2022, the entire disclosures of which are incorporated herein by reference.

### FIELD OF THE INVENTION

The invention generally relates to marketing healthcare information to consumers, providing resources for consumers, including medical services, prescription medications, and other health services. The invention also relates to providing financial services, incentives, and free medications to consumers and or medical practitioners. The invention also relates to patient recruitment for clinical trials and other medical services.

### BACKGROUND

Often times, when a physician prescribes a drug therapy, the pharmacy benefit manager (PBM) or insurance company seeks to change the prescription to a different product. Here, an important motivation of the PBM or insurance company is to receive more money from pharmaceutical companies by controlling (or appearing to control) market share of pharmaceutical products.

With the prevalence and democratization of computers, smart phones, and the internet, consumers seeking medical care and how they seek that care is changing. Consumers are on their computers and the internet now more than ever. Furthermore, consumers are seeking health information not only in content but also in services over the internet.

Telemedicine and telehealth companies are emerging at a faster rate and are becoming more mainstream. Example in point is the changing reimbursement policies of CMS, which tend to favor and promote the use of telemedicine services as a cost containment measure.

Not only are patients seeking relevant medical information and services, pharmaceutical manufacturers continually are seeking appropriate patients for clinical trials and are advertising the products and services that they provide directly to consumers.

Given the steeply increasing cost pressures of health care delivery and new and better medicines, the increasing sophistication of health care consumers, and the ever-growing widespread use and democratization of computers, smart phones, and ready access to information through the internet, there exists a significant need and opportunity for tools, systems, and methods that give patients and pharmaceutical companies more control over drugs costs and distribution.

Disclosed are a system and a method (a) to improve efficiency and cost for pharmaceutical manufacturers to acquire patients that are appropriate for medication therapy and services provided by the pharmaceutical manufacturers, and (2) to improve the efficiency and cost for patients to acquire relevant medical information, services and drugs.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

As pharmaceutical manufacturers are needing a more integrated business model, and patients and practitioners are moving more to telemedicine, the system and method, which are the subject of this disclosure, provide a more efficient way for both the medical practitioner, i.e., healthcare provider, and the pharmaceutical manufacturer to acquire patients, and for patients to have more control of their health care management.

Thus in some aspects, the invention provides a system (upon which the method can be practiced) and a method (which can be executed by the system) for integrating the patient consumer in need of medicine with pharmaceutical manufacturers and healthcare providers (i.e., collectively, the users) and facilitating the communication between the users through the use of a cloud or server-based software application.

Here, telemedicine practitioners, prescription medication programs, and secure payment systems provide patient consumer users with the ability to see a telemedicine practitioner, have appropriate medication prescribed, and receive the medication via a mail order or local pharmacy.

In some embodiments, the system includes, and the method employs a central processing unit, a database or other library of information that includes drugs and indications, and software that enables the exchange of content between a first user and a second user and remote databases and/or programs. Here, the first user or the second user can be the patient consumer, the pharmaceutical manufacturer, or the healthcare provider (including telemedicine provider).

In some embodiments, the system includes, and the method employs a plurality of intercommunicating modules. Those modules may include any two or more of a symptom key, a drug product index, a healthcare provider index, a financial services module, a telemedicine communication module, a clinical trial recruitment module, and a pharmaceutical manufacturers' module.

Many of the attendant features will be more readily appreciated as the same becomes better understood by reference to the following drawings and detailed description.

### DRAWINGS

FIG. 1: pharmaceutical drug information exchange system with multiple modules.

FIG. 2: illustration of system for executing a method for exchanging pharmaceutical drug information between a first user and a second user

FIG. 3 illustration of distributed electronic network that utilizes a distributed computer electronic network communication system for exchanging drug and medical information between a first user and a second user FIG. 4: nested modules, tools, and features accessible by several users

FIG. 5 flowchart for providing drug and drug information to a patient seeking information regarding drug and drug information

FIG. 6: method of providing drug and drug information to a patient through an electronic network distributed system

FIG. 7: system for promoting the informing, prescribing, filling, and tracking of selected pharmaceutical drugs

FIG. 8: flow chart of method for system administrator promoting the informing, prescribing, filling, and tracking of selected pharmaceutical drugs

FIG. 9: flow chart of method for system administrator promoting and reporting filling prescriptions with pharma-promoted drugs

### DETAILED DESCRIPTION

Pharmaceutical manufacturers advertise branded as well as non branded medicines through various media including television, electronic ads, social media, in print, electronic databases, and through live events such as health fairs and the like. Many consumers (patient consumers) may become interested in particular medical, pharmaceutical, or other health-related information, especially if they are concerned about a real or potential medical issue and seek to address those issues.

Disclosed is a system and a method for connecting patient consumers with healthcare providers and/or pharmaceutical manufacturers to obtain medical advice and/or pharmaceutical drugs. Here, a patient consumer queries the electronic system through a user interface, which in turn gathers pertinent patient consumer information from the patient consumer. Patients may also be prompted with questions that are used to screen for specific disease states or treatments, and inclusion into corresponding funded programs. The pertinent consumer information, which can include symptoms, age, sex, geography, medical history, insurance information, and the like, may populate a dynamic database with deidentified personal medical information as part of the overall system.

The electronic system screens and categorizes the inputted patient consumer information and returns advice regarding or a referral to appropriate health care resources, including healthcare provides such as e.g., physician, nurse, prescriber, dentist, chiropractor, and/or the like, or hands-off the patient consumer information to a telemedicine partner that in turn matches the patient consumer to an appropriate healthcare provider. The system also provides the patient consumer an option of choosing a telemedicine provider from a list. The healthcare provider may also be provided with access to special offers relevant to the patient consumer's medical concerns or symptoms.

Here, also, the electronic system may also coordinate or otherwise help provide funding for the patient consumer's healthcare visit and may also provide patient assistance with the medication or treatment prescribed by the healthcare provider. Thus, the system and the method include as users not only patient consumers, but also healthcare providers (including telemedicine partners) and pharmaceutical manufacturers.

The subject system and method provide pharmaceutical manufacturers (sometimes referred herein as “pharma”) with multiple advantages, including an opportunity to find patients appropriate for clinical trials, and securing patient consumers for their drug products without interference from an insurer or PBM changing the original prescribed medication. In some embodiments, the system provides a means for the patient consumer to obtain prior authorization from the PBM or insurer for medications that are not listed on the PBM or insurer's formulary.

The subject system and method provide healthcare providers with multiple advantages, including finding and sending new patients to the provider. Healthcare providers also benefit from the system or method by receiving prepaid visit funding through the system, and/or other incentives offered through the system.

An important aspect of the subject system and method is user friendliness. For example, the system/method is patient consumer user-understandable by providing medicine information in a non-branded way. This means that understandable non-technical medical language is used to present the patient consumer issues and concerns in a therapeutic rather than a branded-name-recognition marketing manner. Here, patient consumers explore their issues by discussing/disclosing their symptoms in common non-technical language that is most commonly understood by them, rather than having to navigate through branded and technical talk. That is, the patient consumer need not refer to a large medical dictionary to discern, for example, the alphabetical identities for diseases (e.g., CPD,GURD,RLS, and the like). However, the use of non-branded language does not preclude the use of branded information. In some embodiments, the system uses branded drug information, such as, e.g., information provided in branded drug commercials and advertisements.

In one aspect, the invention provides a system for exchanging information between a patient consumer and a pharmaceutical company. In one embodiment, the system includes two or more functional modules, a first user interface, a second user interface, a central processing unit, a database of drug and medical indication-related information and healthcare provider information, and one or more object request brokers.

In some embodiments, the two or more functional modules include any two or more of a physical symptom key, a drug product index, a healthcare provider index, a financial services module, a telemedicine communication module, a clinical trial recruitment module, and a pharmaceutical manufacturers' module.

Here, the physical symptom key is accessed and operated via a user interface through which the patient consumer inputs their symptoms. In some embodiments, the physical symptom key provides a questionnaire or interview dialog via the interface to assist the patient consumer with identifying a disease or a syndrome based upon patient-provided information. In some embodiments the patient will be asked if they have specific symptoms to identify a disease or treatment.

Here also, the physical symptom key includes a database or a portal to access a database of diseases and syndromes and their associated symptoms, and a library of approved drugs (and/or drugs currently undergoing clinical trials) for treating the diseases, syndromes, and/or their associated symptoms.

In some embodiments, the system determines whether the symptoms match a specific disease/medication pair supported by a cooperating pharma. In some embodiments, the drug product index includes a database of approved drugs and drugs currently in clinical trials for specific diseases, symptoms or syndromes, which is continually updated or updated on a regular schedule. The database is updated with information provided by government sources (e.g., FDA, Clinicaltrials.gov, and the like) and/or by the pharmaceutical manufactures.

Here, a user can browse the database by inputting a drug name or a symptom, and/or access the database through the physical symptom key user interface. Relevant drug information is presented in an intuitive form to the user via the user interface. In one embodiment, the relevant drug information is presented in a format that includes drug name, brand name, indication, cost, and further information accessible through a hyperlink. Here, relevant means drugs relevant to the symptom query and other patient input information.

In some embodiments, the healthcare provider index includes a database containing information related to physicians or other healthcare providers and telemedicine providers. Here, the module also includes a patient interface with an interview function, wherein the patient inputs symptoms, disease or syndrome identification, and other patient information, including geographic information, into the interface, and the index provides a rank order of healthcare providers relevant to the disease, syndrome, or symptoms, the patient history, and available drugs in relation to the patient information.

In some embodiments, the financial services module provides a portal for paying for healthcare services or medication, as well as for receiving coupons, ordering drugs through special programs, managing grants or loans related to healthcare, and/or receiving payments for participation in clinical trials. Here, the financial services module includes any one or more of a payment selection and enablement tool, a coupon selection tool, a drug sample request form, a discount request tool, a database of approved drugs and discounts applied thereto, and a database of available clinical trials related to specific drugs and patient participant payments related thereto.

In some embodiments, the telemedicine communication module enables the communication between the patient consumer and the telemedicine partner, and/or the healthcare provider to effect inter alia diagnosis of the patient's symptoms/disease, prescribing medicine, and general medical counseling by the licensed healthcare provider. Here, the telemedicine communication module provides any one or more of text, chat, audio, screen sharing, and video chat tools to permit real-time exchange of information between patient consumer and telemedicine practitioner, in addition to supporting live interactive video, store-and-forward transmission, remote patient monitoring, and mobile health platforms.

In some embodiments, the clinical trial recruitment module enables the efficient recruitment of patient consumer participants to clinical trials sponsored by the pharmaceutical manufacturer, in part by providing patient consumer users with information regarding clinical trials for drugs relevant to their diagnosis, which was made by a licensed healthcare provider, and/or symptoms, accessed through the patient consumer user interface. Here, the clinical trial recruitment module includes any one or more of (a) a participant recruitment, registration, and tracking tool, (b) HIPAA secure medical records and/or genetic information of trial participants, including the patient consumer, (c) a payment tool for remunerating participants, which is also included in the financial services module, and (d) a database of currently recruiting clinical trials, which includes inter alia information for particular drugs, indications, subject patient attributes, geographic area or clinical trial centers, and general subject participant criteria.

In some embodiments, the manufacturers' module includes an interface for the pharmaceutical manufacturer user to access patient consumer and/or healthcare provider information and to interact with the patient consumer and/or healthcare provider users. Here, the manufacturers' module includes a database or library of information including patient consumer user information (which may be deidentified or include a secure waiver) that is cross referenced to symptoms, syndromes, or diseases. Here also, the manufacturers' module includes a database or library of information including healthcare provider information, including telemedicine provider information. The information may be cross referenced to geography, specialty, licensing, and practice size or availability.

In some embodiments, any one or more of the aforementioned modules includes a software tool that enables or facilitates communication with one or more distributed network programs. Here, the distributed network programs operate over a distributed networking system of processes, threads, agents, and distributed objects, employing concurrent program execution. In some embodiments, the distributed networking system operates according to or including any one or more of a client/server system, an agent-based system, a decentralized system, and a mesh networking system.

In another aspect, the invention provides a method for exchanging information between a patient consumer and a pharmaceutical company. In one embodiment, the method includes the steps of a first user accessing a remote program or database from a first user interface, and then uploading or downloading user information from the program or database. A second user also accesses the remote program or the remote database from a second user interface, and then downloads information that was uploaded by the first user and then subsequently acts on that information. In one embodiment, the remote program or database is accessed by each of the users via their respective interfaces through a system using distributed computer middleware, as described above. Here, information is exchanged between and among the users through the system.

In some embodiments, the first user is a patient consumer and the second user is a pharmaceutical manufacturer, the first user is a pharmaceutical manufacturer and the second user is a patient consumer, the first user is a patient consumer and the second user is a healthcare provider, the first user is a healthcare provider and the second user is a patient consumer, the first user is a healthcare provider and the second user is a pharmaceutical manufacturer, or the first user is a pharmaceutical manufacturer and the second user is a healthcare provider.

In some embodiments, the method includes exchanging information among three or more users, including any three or more of a patient consumer, a pharmaceutical manufacturer, a healthcare provider, a system manager, and optionally, a government agency, such as the Food and Drug Administration, the National Institutes of Health, the Centers for Disease Control and Prevention, or other agency in the Department of Health and Human Services. In a preferred three-or-more-user embodiment, the users include a patient consumer, a pharmaceutical manufacturer, and a healthcare provider.

In some embodiments, the method employs or the users access one or more databases including a database containing diseases and syndromes and their associated symptoms and approved drugs for treatment, a database of de-identified patient consumers cross-referenced to their related symptoms, syndromes, or diseases, a database of approved drugs and drugs currently in clinical trials for specific diseases, symptoms or syndromes, a database of physicians or other healthcare providers and telemedicine providers cross-reference to specialty, licensing, and geography, a database of approved drugs and discounts applied thereto, a database of available clinical trials related to specific drugs and patient participant payments related thereto, a database of currently recruiting clinical trials for particular drugs, indications, subject patient attributes, and geographic area, and a database of healthcare providers, including telemedicine providers, cross referenced to geography, specialty, licensing, and practice size or availability.

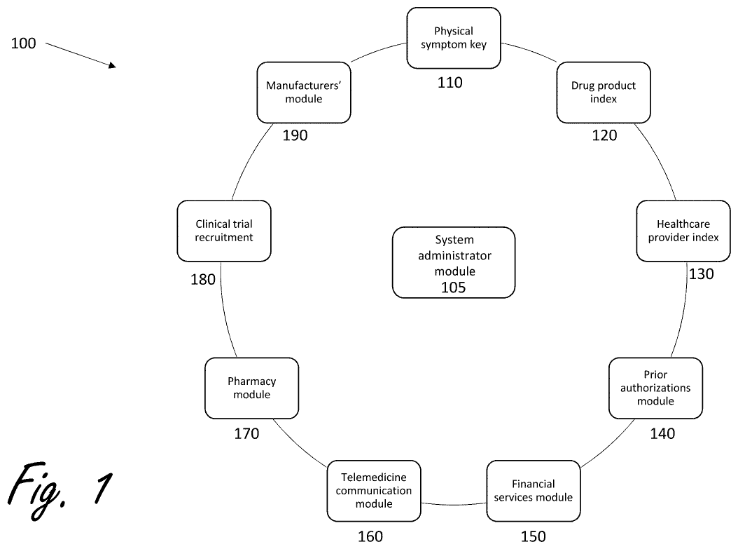

Turning to FIG. 1, in one aspect, the invention provides a pharmaceutical drug information exchange system 100. In one embodiment, the system is located and employed across a distributed networking system that includes user interface “nodes” for user access to the system, several functional modules within the system 100, and a system administrator module 105, through which the system administrator manages the system 100, facilitates interactions and communication between the modules of the system, coordinates system activity, and promotes streamlined and efficient delivery of pharmaceutical manufacturer-sponsored drugs to pharmacies and patients. Here, the pharmaceutical drug information exchange system 100 includes a physical symptom key 110, which interfaces with the patient consumer user to enable the user to “key-out” or identify symptoms using common non-technical language; a drug product index 120, which includes a searchable or query-based database of drug product information by indication and availability, which is regularly updated with information provided by a healthcare user or a pharmaceutical manufacturer user and managed by the system manager; a healthcare provider index 130, which includes a searchable or query-based database of healthcare provider (pharmacy, physician, nurse, physician's assistance, telemedicine provider, pharmacist, and the like) by medical specialty, geography, and availability; a financial services module 150, which enables the exchange of funds (both payments to a patient consumer user for use to buy medicines or in exchange for participating in a clinical trial, and payments from a patient consumer user for the purchase of medicines through the system), coupons, discounts, and drug samples, not unlike a VENMO or ZELLE type payment exchange module, preferably, where the funds are used by a patient consumer to fill a prescription, via a secondary copayment card that can be tracked by the system administrator or the pharma sponsor; a prior authorizations module 140 for enabling securing by a prescriber prior authorizations for non-formulary drugs; a telemedicine communication module 160 for accommodating information exchange between the patient consumer user and the healthcare provider user for scheduling patient visits, video chats, messaging between healthcare provider and patient, exchange of test results and medical records, and the like; a pharmacy module 170 for a pharmacy, which can be a mail-order pharmacy and/or a bricks and mortar pharmacy, to engage with the system and system components, especially as it relates to filling prescriptions for drugs sponsored by the pharmaceutical manufacture for patient consumers, and delivering discounts, samples, and the like as available or necessary. Here, the pharmacy and the system administrator communicate within the system to exchange information such as, e.g., prior authorization information for a patient consumer to obtain a pharmaceutical manufacturer-sponsored drug that may not be on the patient consumer's insurance company's formulary for a particular indication, and other insurance company cum patient information. Here also, communications between the pharmacy and a healthcare provider (via the healthcare provider index 130, the telemedicine communication module 160, and/or the pharmacy module 170) are enabled and facilitated.

Here also, the system 100 includes a clinical trial recruitment module 180 for the recruitment of trial participants by healthcare providers or pharmaceutical manufacturers; and a pharmaceutical manufacturers' module 190, which enables the pharmaceutical manufacturer user to access patient consumer and/or healthcare provider information and to interact with the patient consumer and/or healthcare provider users, especially for promoting patient consumer-relevant drugs sample, clinical trial products, or medications to an assigned pharmacy in the system, be it a mail order or a local pharmacy. Here, the patient consumer may be eligible to receive these samples, clinical trial products, or medications a no charge or supplemented price. The pharmaceutical manufacturers' module 190 also enables communication with the pharmacy module 170 to facilitate the exchange of information, samples, free goods, program drugs, and the like, between the pharmaceutical manufacturer and the pharmacy.

In a preferred embodiment, the free or discounted prescription is automatically provided by the system administrator to the healthcare or telemedicine provider.

In some embodiments, the modules are functional modules that share at least one tool or database between two or more modules.

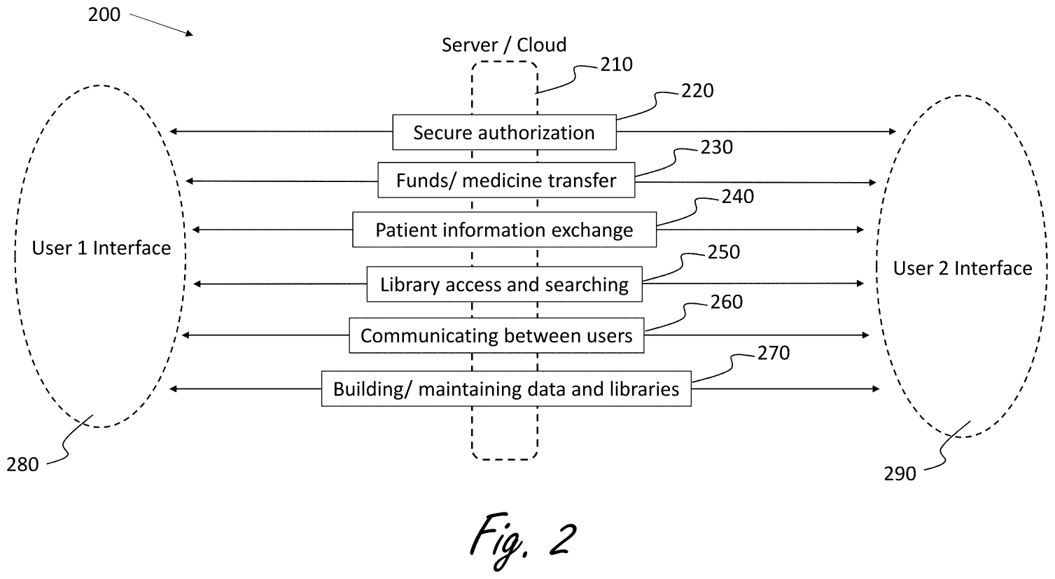

Turning to FIG. 2, in one aspect, the invention provides a system 200 for executing a method for exchanging pharmaceutical drug information between a first user 280 and a second user 290 and obtaining pharmaceutical drugs. In one embodiment, the system 200 includes a first user interface 280, a second user interface 290, and a server/distributed network program 210. Here, the user interfaces 280/290 can be any electronic interface such as a computer, laptop, tablet, smart phone or the like that allows for the entering (uploading) and receiving (downloading) of information from the server/distributed network 210 by the user.

Here, information is exchanged between the first user 280 and the second user 290 via the server/distributed network 210. In one embodiment, each server accesses the system 200 by entering security credentials (thumbprint, facial recognition, password, passcode, two-step authorization, or the like) to obtain secure authorization 220 to access the system 200. In some embodiments, the first user is a patient consumer and the second user is a pharmaceutical manufacturer, the first user is a pharmaceutical manufacturer and the second user is a patient consumer, the first user is a patient consumer and the second user is a healthcare provider, the first user is a healthcare provider and the second user is a patient consumer, the first user is a healthcare provider and the second user is a pharmaceutical manufacturer, or the first user is a pharmaceutical manufacturer and the second user is a healthcare provider. Other users may include the system manager, insurance company/PBM, and/or mail order or bricks and mortar pharmacy.

In one embodiment, funds are exchanged 230 between the first user and the second user for products (i.e., proprietary information, prescribed drugs, drug samples, and the like) or money (i.e., payment for clinical trial participation, payment for drugs, monetary grants, loans, or coupons for obtaining medicine).

In one embodiment, patient information is exchanged 240 between the patient consumer user and the healthcare provider, pharmaceutical manufacturer, pharmacy, and/or system manager. Here, patient information includes patient symptoms input by the patient consumer user, personal information such as e.g., age, weight, sex, ethnicity, medical history, insurance, other health benefits or liabilities, and/or the like, which is accessed by the second user, such as a licensed healthcare provider, for inter alia diagnosis and/or prescribing purposes.

In one embodiment, any one or more users accesses and/or searches a database or library of information 250. Here, the databases are directed to inter alia (a) drug information, such as e.g., drug indications, drug contraindications, current trials for drugs, drug cost and availability, and the like, (b) healthcare provider information, and/or (c) patient information, including deidentified medical histories, genetic information, waived protected health information, and the like.

In one embodiment, the first user 280 and the second user 290 communicate with each other 260 through the server/distributed network 210 within the system 200. Such communicating includes e.g., messaging, video chatting, calendar sharing, and the like.

In one embodiment, any user, through their interface 280/290, including the system manager, pharmaceutical manufacturer, healthcare provider, and/or pharmacy provides information that is used to build and maintain each database and library of information 270 to ensure that the databases and libraries are current and accurate. In one embodiment, the system manager annotates, collates, organizes, and serves as a gate-keeper or quality control manager of the updating information.

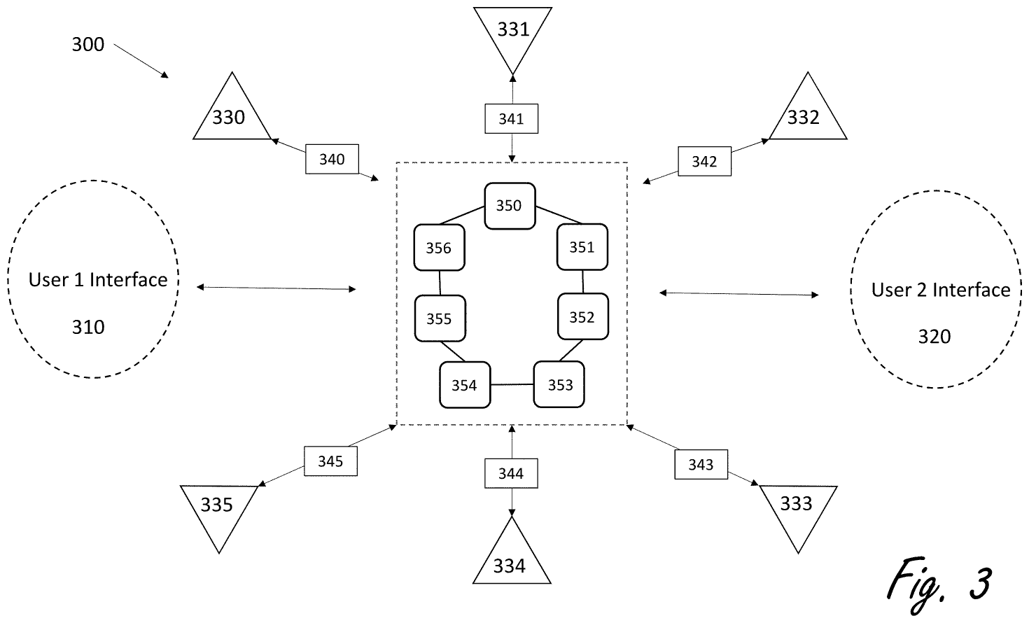

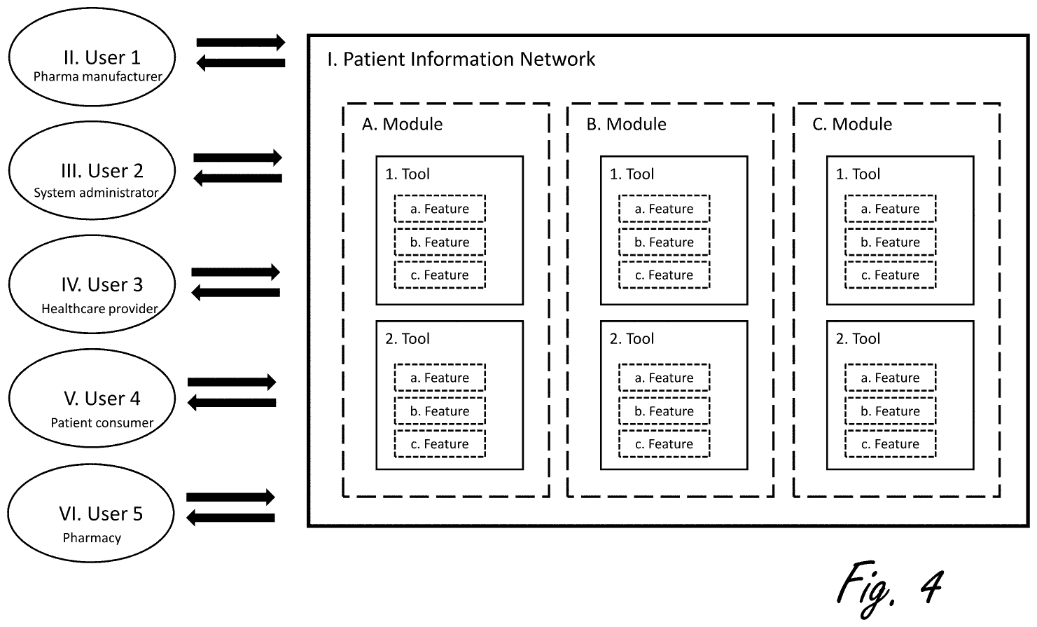

Turning to FIG. 3, in one aspect, the invention provides a distributed electronic network that utilizes a distributed computer electronic network communication system 300 for exchanging drug and medical information between a first user and a second user. In some embodiments, the first user is a patient consumer and the second user is a pharmaceutical manufacturer, the first user is a pharmaceutical manufacturer and the second user is a patient consumer, the first user is a patient consumer and the second user is a healthcare provider, the first user is a healthcare provider and the second user is a patient consumer, the first user is a healthcare provider and the second user is a pharmaceutical manufacturer, or the first user is a pharmaceutical manufacturer and the second user is a healthcare provider.

Other users may include the system manager, insurance company/PBM, and/or pharmacy. Here, in some embodiments, the first user is a patient consumer and the second user is any one of the system manager, the insurance company, and the pharmacy; the first user is a pharmaceutical manufacturer and the second user is any one of the system manager, the insurance company, and the pharmacy; the first user is a healthcare provider (including telemedicine provider) and the second user is any one of the system manager, the insurance company, and the pharmacy; the first user is a system manager and the second user is any one of the insurance company and the pharmacy; and/or the first user is an insurance company and the second user is the pharmacy. In one embodiment, pharmacy data may be communicated to the pharmaceutical manufacturer via the system administrator.

The system 300 includes a first user interface 310, a second user interface 320, functional modules 350-356 as described in FIG. 1, remote software programs and/or databases as graphically depicted as elements 330-335, and concomitant distributed computing middleware programs, such as, e.g., object request brokers (ORB) to permit calls to be made from one computer to another via a computer network and to permit communication amongst disparate objects, as graphically depicted as elements 340-345. Here, the number of graphically depicted middleware programs 340-345 -250, modules 350-356, and software/databases 340-345 useful in the practice of the invention are not limited to the number of graphical depictions in FIG. 3.

Turning to FIG. 4, in one aspect, the invention provides a system 400 that contains nested modules, tools, and features, which are accessed by several users through various controls, filters and access privileges. In one embodiment, each user 1-5 has a distinct function and gains value by interacting thorough the system with one or more other users of the system. In a preferred embodiment, user 1 is a pharmaceutical manufacturer; user 2 is a system manager; user 3 is a health care provider; user 4 is a patient consumer, and user 5 is a pharmacy (mail order and/or bricks and mortar).

In one embodiment, the system (preferably a patient information network system) includes more than two modules, such as, e.g., module A, module B, module C, et seq. Each module includes one or more tools (e.g., tool 1, tool 2, et seq.). Specific tools may be used by more than one module. In some embodiments, each tool includes one of more features (e.g., feature a, feature b, feature c, et seq.). Specific features may be used by more than one module or tool.

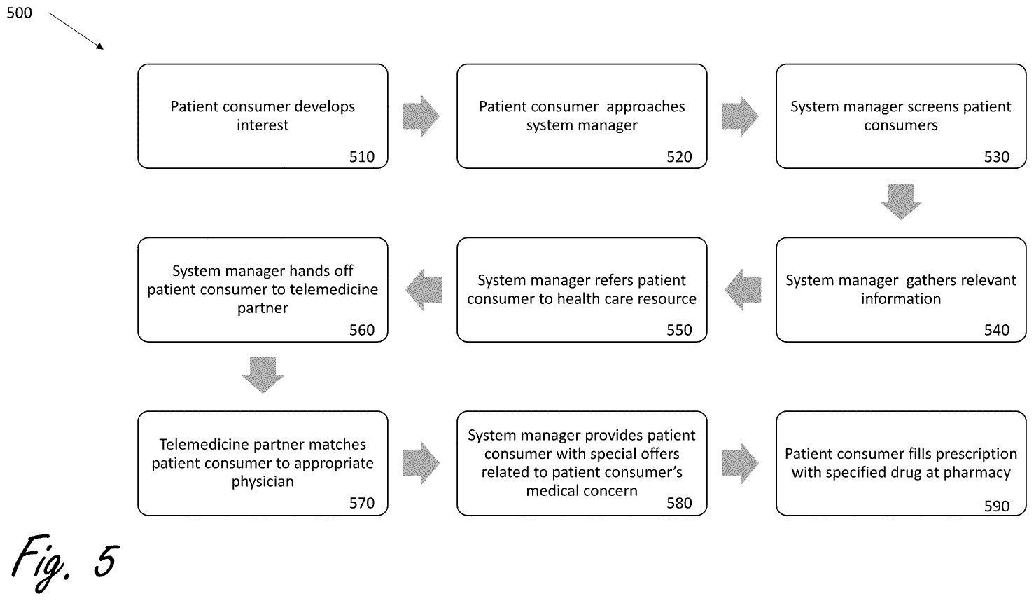

Turning to FIG. 5, in one aspect, the invention provides a method 500 for providing drug and drug information to a patient seeking information related thereto. In one embodiment, a patient consumer (a first user) develops interest 510 in learning about treatment options for particular symptoms or disease. The patient customer then approaches the system manager 520. Here, the system manager is a second user that interfaces with the first user patient customer through a network distributed system. The system manager then screens the patient consumer 530 and gathers relevant information 540 by interviewing the patient consumer and reviewing the input patient information through the system. In one embodiment, the patient consumer may opt into the system (or, conversely, opt out of the system).

Based upon its evaluation of the relevant information, the system manager refers 550 and hands-off 560 the patient consumer to a healthcare provider or telemedicine service (a third user). In some embodiments, the healthcare provider for the patient may be selected by the patient, the system manager, or the telemedicine partner.

In one embodiment, where the patient consumer is referred to a telemedicine partner, the telemedicine partner refers 570 the patient consumer to an appropriate physician. Here also, the system manager then provides 580 the patient consumer with special offers related to the patient consumer's particular medical concerns. Those special offers may include, e.g., drug samples, drug purchase coupons, financial assistance for particular drugs, secondary copay cards, the opportunity to participate in a related clinical trial, and the like. Further, the patient consumer and the healthcare provider than communicate with each other via the subject network distributed system described herein. In some embodiments, the patient consumer uses the special offer (e.g., coupon, copay card, and/or the like) to obtain a prescription at a pharmacy 590.

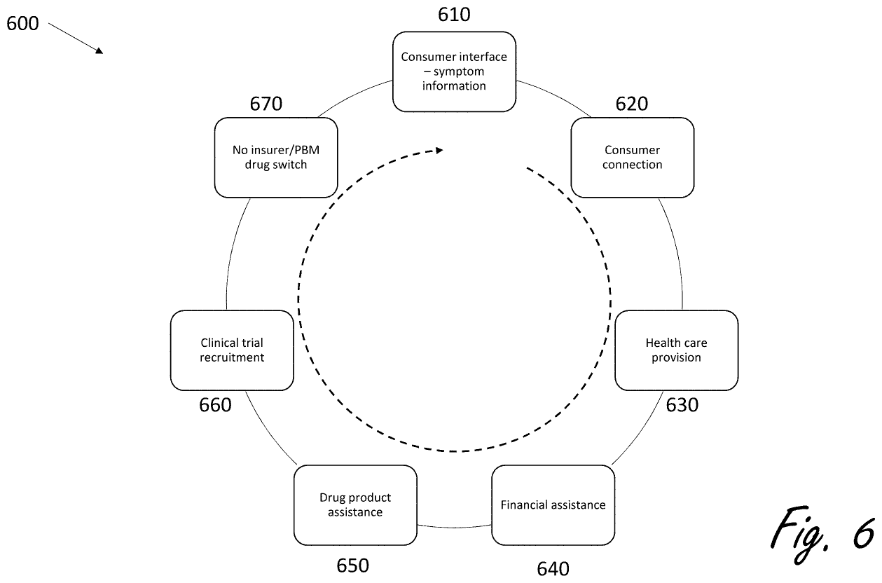

Turning to FIG. 6, in one aspect, the invention provides a method 600 for providing drug and drug information to a patient seeking information related thereto through an electronic network distributed system. In one embodiment, a patient consumer provides 610 symptom information through a user interface into the system. Next, the patient consumer connects 620 via the system to a healthcare provider, a system manager, or a pharmaceutical manufacturer. A healthcare provider evaluates the patient consumer information through the system and then provides 630 healthcare advice to the patient consumer through the system. In some circumstances and as needed, the patient consumer receives drug product assistance 650 or other financial assistance 640 from either the healthcare provider or the pharmaceutical manufacturer for obtaining the required medication. Such assistance can be in the form of forgivable loans, grants, coupons, drug samples, and the like.

In some embodiments, the healthcare provider, system manager, and/or pharmaceutical manufacturer recruits or offers to recruit 660 the suitable patient consumer into a suitable clinical trial, thereby providing access to the patient consumer of relevant drugs through the clinical trial.

In some embodiments, the patient consumer is offered the relevant and preferred drug through the system without any input or other interference from an insurer or PBM, thereby preventing drug switching 670. Here, the pharmaceutical manufacturer and the patient consumer control the drug selection and delivery process outside of another entity, such as an insurer or PBM.

In one aspect, the invention provides a system and method for promoting the informing, prescribing, filling, and tracking of selected pharmaceutical drugs (a.k.a., pharmaceuticals, drugs, medicaments).

In one embodiment, the system is an open network system, meaning that the system is open to enlistment or participation by any patient or provider regardless of the insurance plan or PBM the patient belongs or participates in. While the system is an open network as it relates to patient participation, the system is a closed system regarding the exchange of information, goods, and services among the pharmaceutical manufacturer, the patient consumer, the healthcare provider or telemed prescriber, and the pharmacy. This closed system allows the pharmaceutical manufacturer to track the effectiveness of their marketing (e.g., television commercials, radio commercials, print ads or electronic ads) and to follow the patient consumer through the process of receiving a prescription and filling that prescription. For example, the pharmaceutical manufacturer may obtain information such as how many prospective patient consumers responded to the marketing program, how many of those prospective patient consumers contact the system administrator, how many patient consumers completed a telemedicine visit, how many patient consumers were prescribed a program drug 9 a.k.a. pharma-sponsored drug), how many patient consumers obtained the program drug, and when, where, and how often are the prescriptions filled.

In one embodiment, the patient consumer can qualify to participate in the system, which is a single, closed, and continuous system (a.k.a. the program), and receive information about health conditions, healthcare providers, and medicine all within the continuity of the single system. In other words, in one embodiment, a patient may view or hear a pharmaceutical drug advertisement or offer through a commercial or other advertisement, but their physician/healthcare provider is not yet aware of the drug; or there is no funding available to the patient for the doctor visit; or the patient does not know where the medication is available, i.e., by which pharmacy. Here, in one specific embodiment, the prospective patient consumer contacts the system administrator based on information provided in the commercial or other advertisement, the system administrator obtains information form the patient (e.g., insurance information and the like) and determines whether the prospective patient is suitable for the program, the system administrator refers the patient consumer to a telemedicine healthcare provider, the healthcare provider examines the patient and prescribes a suitable program drug and forwards to prescription to a program mail-order pharmacy to be filled, the patient applies a program discount, copay card (electronic or physical card), or the like received by the patient from the system administrator, and the prescription is filled and mailed to the patient. Here, in whole or in part, information regarding this pharmaceutical drug transaction is available for the edification of the pharmaceutical manufacture to track its drug sales and the In some cases, some information regarding the transaction may not be readily available to the pharmaceutical manufacturer due to insurance company or pharmacy patient privacy concerns and considerations. Here, upon entering, joining, or otherwise engaging with the subject closed system, the prospective patient consumer is given the opportunity to opt in to the system by authorizing a health information waiver, or by not declining an automatic waiver, thereby permitting their patient health information to be shared with the pharmaceutical manufacturer as part of the pharmaceutical drug transaction information package available to the pharmaceutical manufacturer.

In some embodiments, information is provided to potential patient consumers about specific symptoms and corresponding disease states and the potential associated medications targeting those indications. In some embodiments, the drug messaging can take any form, such as, e.g., television or radio advertisements, online advertisements, Google keyword matching, and/or the like. Here, the messaging form includes links, short code texting, and/or the like, that brings the potential patient consumer to a screening process that checks whether the patient has symptoms of a specific ailment that can be treated by a specific medication. This screening determines whether a next step is taken with the potential patient consumer, such as setting up a telemedicine (a.k.a. telemed) appointment. Here, if the patient consumer passes the screening, then a telemed appointment is arranged and provided with financial assistance provided from the system administrator, including, in some embodiments, discounted or free first medication. Here, the system administrator obtains funding to provide this financial assistance from a pharmaceutical manufacture (a.k.a. pharma or pharma sponsor). In a preferred embodiment, the system administrator encourages and facilitates the prescribing and filling of a drug manufactured and/or sold by the pharma sponsor.

Thus, in one embodiment, the system administrator or the healthcare/telemed provider provides the patient consumer with a secondary copay card to be used to purchase the pharma sponsor-recommended drug. When the patient consumer fills or refills the prescription and applies the secondary copay card, the transaction is tracked, and information collected and reported to the pharma sponsor. In another embodiment, the prescription sent from the telemed prescriber to the assigned pharmacy, which may be mail order or a local pharmacy, and that pharmacy provides the medication at no cost to the patient consumer. Here, communication proceeds between the system administrator and the pharmacy, and whereby the pharmacy receives its renumeration from the system administrator and/or the pharmaceutical manufacturer by any means, such as and including a copay card, which may be in the form of a physical card or of an electronic “card.”

In one embodiment, the system administrator employs screening tools to determine whether a potential patient consumer is appropriate for further engagement in the system. In a preferred embodiment, the screening tools include a battery of specific questions that the pharma sponsor provides to the system administrator to determine whether the potential patient consumer matches (or largely matches) the criteria for the ailment that their medication would be prescribed for.

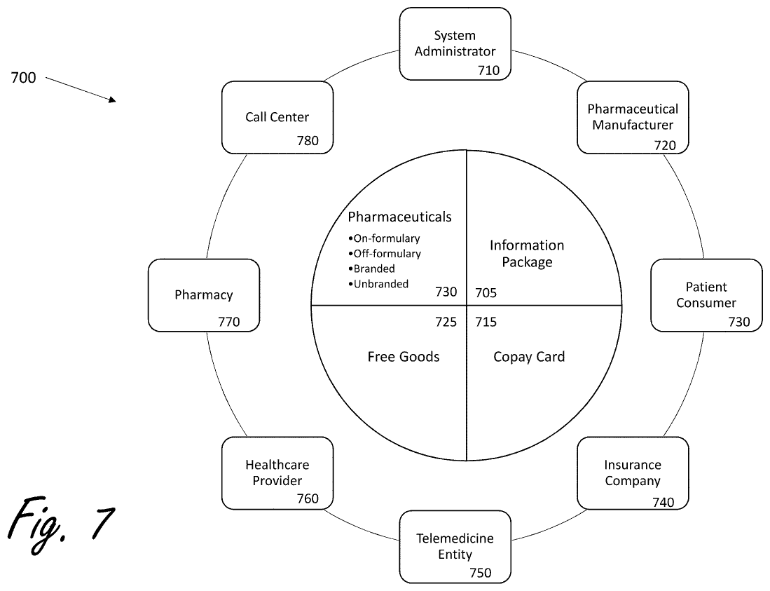

Turning to FIG. 7, in one aspect, the invention provides a system 700 for promoting the informing, prescribing, filling, and tracking of selected pharmaceutical drugs. In one embodiment, the system includes a system administrator 710, which, in some embodiments, serves to coordinate the activities and communications between and among the other components of the system 700, and which, in some specific embodiments, may employ a third-party PBM or operate as a PBM while administering the system; a pharmaceutical manufacturer (pharma or pharma sponsor) 720, which, in some embodiments, subscribes to the system and contributes funding, screening tools, an information package 705, which includes the advertisement information presented to the potential patient consumer 730 as well as information employed in the screening tools used by the system administrator 710, and the like to the system, and receives information back regarding drug prescription and filling metrics; a patient consumer 730; optionally an insurance company 740, which, in some embodiments, provides authorization or prior authorization for those pharma-sponsored drugs 735 that may not be included on the insurance company's formulary; a telemedicine entity (telemed or telemed provider) 750, which, in some embodiments, is selected by the system administrator, trained to operate its role within the system, and is provided with funding for copay cards 715, discounts, free goods 725, such as sample drugs and program drugs, and/or the like to assist and facilitate the patient consumer to acquire the selected prescribed pharma-sponsored drug; a healthcare provider (e.g., physician or other licensed individual legally competent to prescribe drugs 735) 760, which, in some embodiments, is provided with funding for copay cards, discounts, and/or the like to assist and facilitate the patient consumer to acquire the selected prescribed pharma-sponsored drug 735; a pharmacy (e.g., mail order or bricks and mortar) 770, which, in some embodiments, fills or refills the prescribed pharma-sponsored in exchange for, in part or in whole, payment presented via a copay card 715; and a call center or other information-repository 780 that is accessed by another element of the system, preferably by the potential patient consumer, who is responding to the information package 705, which was preferably presented in an advertisement, by calling or texting a number or link provided by the advertisement information or other information package 705.

In some embodiments, the system 700 operates across a distributed network system, preferably a distributed network system that incorporates electronically conveyed information (the information package 705), computer interface devices used by the patient consumer (e.g., tablets, notebooks, mobile phones, desktop computers, and the like), central processing units, such as a server located on the cloud or locally, for storing and accessing the information package 705, which can be in the form of a database, and operating software across the system, all connected via data transmission media, such as a telecommunication circuits, an ethernet circuits, wireless fidelity (WiFi), a microwave circuits, ultrahigh frequency (UHF) radio transmissions, other radio transmissions, and/or the like.

In some embodiments, the copay card 715 is issued to the screened and approved patient consumer 730 by the system administrator 710. Here, the copay card 715 can be a secondary copay card used by the patient consumer 730 to pay (in part or in whole) for the pharmaceuticals 735 that are prescribed by the telemed 750 or healthcare provider 760. Preferably, the copay card 715 is used to purchase pharma-sponsored pharmaceuticals. Regardless of whether the copay card 715 is used to purchase a pharma-sponsored pharmaceutical, by using the copay card 715, the system administrator 710 and/or the pharmaceutical manufacturer 720 can track the purchases made through the card 715 and collect data regarding the filling of prescriptions, particularly what drugs are being used to fill the prescription at the pharmacy 770 and at what frequency and from what healthcare provider 760 and insurance company or PBM 740.

In some embodiments, the pharmaceutical drugs 735, such as the pharma-sponsored drugs, may be listed on the insurance company/PBM 740 formulary, not listed on the insurance company/PBM 740 formulary, a branded drug, or an unbranded drug.

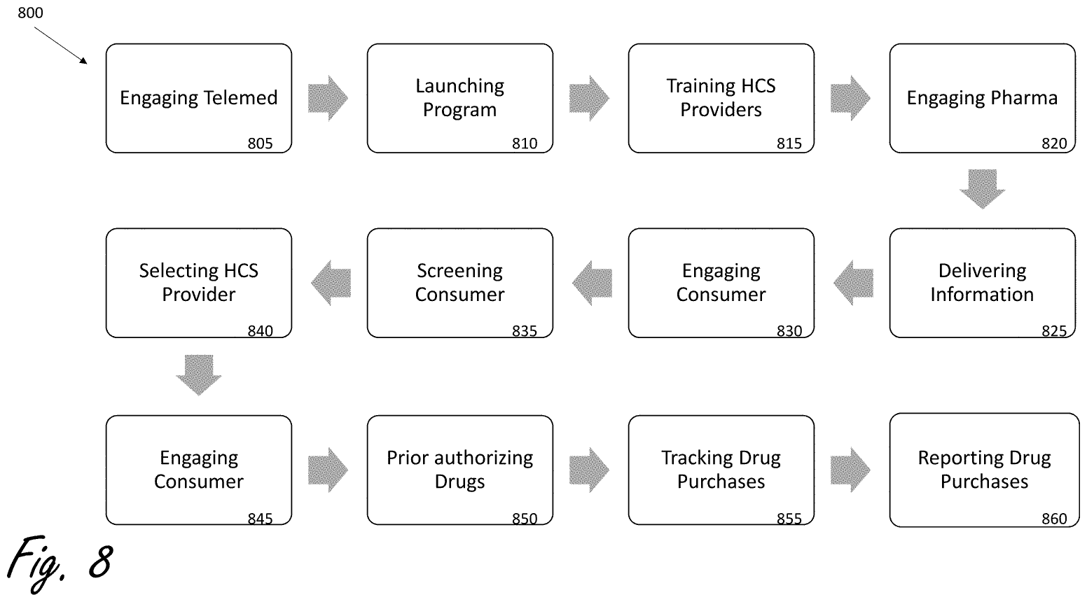

Turning to FIG. 8, in one aspect, the invention provides a method Boo for promoting the informing, prescribing, filling, and tracking of selected pharmaceutical drugs. In one embodiment, the method Boo includes the steps of the system administrator engaging a telemedicine provider 805 to participate in the program for preferentially prescribing pharma-sponsor pharmaceutical drug; the system administrator launching the program 810 for informing, prescribing, filling, and tracking of selected pharmaceutical drugs; the system administrator training healthcare service providers (which may be included within or overlapping with the set of telemedicine provider) 815 in how to use or engage with the program; the system administrator engaging with a pharmaceutical manufacturer 820, which includes onboarding the pharmaceutical manufacturer into the system or on the program, arranging to embed system or program call-in information into drug marketing information of the pharmaceutical manufacturer, and obtaining funds from the pharmaceutical manufacturer to finance coupons, discounts, copay instruments to enable the system or program; delivering information 825 to the prospective or current patient consumer, which contains prescribing information, indication symptoms, patient criteria, and other like information related to the drug (preferably branded), and information on how the prospective patient can contact the system administrator to learn more about the drug and to enable the system provider to screen the patient consumer, such information preferably delivered via a radio or television commercial, internet or social media ads, links, or banners, and the like; the potential patient consumer, having received the delivered information 825, contacting the system administrator by way of the means provided in the delivered information, and the system administrator engaging the prospective patient consumer 830; the system administrator screening the prospective patient consumer (prospect) 835 by interviewing the prospect, either in writing, electronically, orally or otherwise, preferably using questions and/or screening tools provided by the pharmaceutical manufacturer, to determine whether the prospect is a viable candidate to receive the pharma-sponsored drug; the patient consumer selecting a healthcare service provider 840, which includes telemedicine providers, preferably pre-selected and training in the system or program by the system administrator; the healthcare provider or telemedicine provider engages the patient consumer 845 to determine using reasonable and competent medical practice the appropriateness of prescribing a subject pharma-sponsored drug and prescribing a subject pharma-sponsored drug to the patient consumer if medically necessary or otherwise appropriate to be submitted to and filled by the pharmacy. Preferably, the prescription is electronically submitted to the pharmacy, and more preferred, where the pharmacy is a mail order pharmacy.

In some embodiments, a subject pharma-sponsored drug may not be listed on the patient consumer's insurance company or PBM formulary. Here, when the use of the subject pharma-sponsored drug is warranted by the licensed healthcare provider to be medically necessary or preferred, the system administrator facilitates, enables, or otherwise assists the healthcare provider in obtaining prior authorization from patient's insurance company/PBM 850, such as by pre-filling, completing, or otherwise readying the requisite forms for authorization by the licensed healthcare provider (e.g., electronic or physical signature of the licensed healthcare provider or provider's authorized agent).

Once the prescription for the subject pharma-sponsored drug is made, the patient consumer receives a copay card from the healthcare provider to pay in whole or in part for the subject pharma-sponsored drug. In some embodiments, the copay card provides a cash equivalent and/or embodies a discount coupon provided by the pharmaceutical manufacturer and managed or administered by the system administrator. Here, the patient takes the prescription for the subject pharma-sponsored drug to the pharmacy for filling, and then tenders the copay card, which provides a cash equivalent and/or a pharma-provided discount, to purchase the drug. In another embodiment, the system pharmacy provides drug product at no charge to the patient consumer. Here, the pharmacy may receive samples or program medication from the pharmaceutical manufacturer at no charge for use with program patient consumers; and may receive funds directly from the system administrator for fees and services as rendered.

In some embodiments, the system administrator and/or pharmaceutical manufacturer track the filling of the prescriptions and drug purchases 855 through the use of the copay card. Thus, any drug purchases made with the copay card are reported 860 to the system administrator and/or pharmaceutical manufacturer along with ancillary useful information. Other embodiments include free fill at the mail-order-pharmacy may have samples/program drugs at no or supplemental cost.

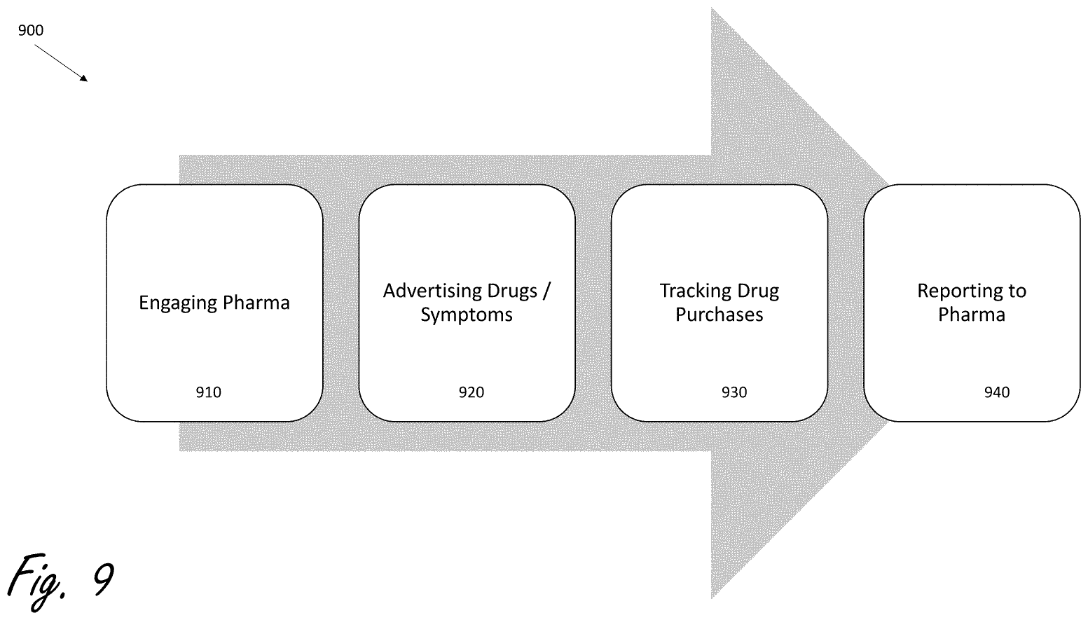

Turning to FIG. 9, in one aspect, the invention provides a system and method 900 for promoting and reporting filling prescriptions with pharma-promoted drugs. In one embodiment, the method 900 includes engaging pharma 910. Here the system administrator contracts a pharmaceutical company to participate in the system or program to promote their products by providing funding to the system administrator, part of which is used for administrative and service providing use and profits, and part of which is used to provide discounts and copayment funds for a patient to use to purchase prescribed medications.

The pharmaceutical manufacturer, preferably consulting with the system administrator, presents marketing information 920 to prospective patients. Here, the information is preferably presented in the form of a commercial or advertisement that describes symptoms, ailments, diseases or syndromes to which a pharma-sponsored drug may be appropriate. The information also includes information on how the prospective consumer can learn more about the drug and whether they are eligible for discounts, copay funds, or other financial relief toward purchasing the drug.

If the patient consumer is potentially eligible to receive the drug, they select or are referred to a healthcare provider or telemed and prescribed the drug by a licensed prescriber if appropriate. In one embodiment, the patient consumer receives a copay card along with the prescription to be used when filling the prescription. Whenever the patient consumer used the copay card to purchase a drug, the pharmaceutical manufacturer and/or system administrator receives a report 940. In some cases, the pharmacy dispenses samples and or program drugs from the system pharmaceutical manufacturer to the patient consumer. Here, the pharmaceutical company can promote the preferred prescribing and sale of its drug and track the purchasing patterns of patients.

The foregoing description and other objects are intended to be illustrative of the invention and are not meant in a limiting sense. Many possible embodiments of the invention may be made and will be readily evident upon a study of the following specification and accompanying drawings comprising a part thereof. Various features and combinations of invention may be employed without reference to other features and combinations. Other objects and advantages of this invention will become apparent from the following description taken in connection with the accompanying drawings, wherein is set forth by way of illustration and example, an embodiment of this invention and various features thereof.

1. A system for exchanging information between a patient consumer and a pharmaceutical company, the system comprising two or more functional modules, a first user interface, a second user interface, a central processing unit, a database comprising drug and medical indication-related information, and one or more object request brokers.

2. The system of claim 1, wherein the two or more functional modules are selected from the group consisting of a physical symptom key, a drug product index, a healthcare provider index, a financial services module, a telemedicine communication module, a pharmacy module, a clinical trial recruitment module, and a manufacturers' module.

3. The system of claim 2, wherein the physical symptom key comprises an interface for inputting symptoms, a questionnaire to assist with identifying a disease or a syndrome based upon patient-provided information, and a database containing diseases and syndromes and their associated symptoms and approved drugs for treatment.

4. The system of claim 2, wherein the drug product index comprises a database of approved drugs and drugs currently in clinical trials for specific diseases, symptoms or syndromes, and a patient interface with an interview function, wherein the patient inputs symptoms and other patient information into the interface, and the index provides a rank order of drugs and investigational drugs relevant to said symptoms in relation to the patient information.

5. The system of claim 2, wherein the healthcare provider index comprises a database of physicians or other healthcare providers and telemedicine providers, and a patient interface with an interview function, wherein the patient inputs symptoms, disease or syndrome identification, and other patient information, including geographic information, into the interface, and the index provides a rank order of healthcare providers relevant to said disease or syndrome and available drugs in relation to the patient information.

6. The system of claim 2, wherein the financial services module comprises a payment selection and enablement tool, a coupon selection tool, a drug sample request form, a discount request tool, a database of approved drugs and discounts applied thereto, and a database of available clinical trials related to specific drugs and patient participant payments related thereto.

7. The system of claim 2, wherein the telemedicine communication module provides (a) text, chat, audio, screen sharing, and video chat tools to permit real-time exchange of information between patient consumer and telemedicine practitioner, and supports (b) live interactive video, store-and-forward transmission, remote patient monitoring, and mobile health platforms.

8. The system of claim 2, wherein the clinical trial recruitment module comprises (a) a participant recruitment, registration, and tracking tool, (b) HIPAA secure medical records and genetic profiles of participants, (c) a payment tool for remunerating participants, and (d) a database of currently recruiting clinical trials for particular drugs, indications, subject patient attributes, and geographic area.

9. The system of claim 2, wherein the manufacturers' module comprises (a) a database of patients, cross referenced to symptoms, syndromes, or diseases, and (b) a database of healthcare providers, including telemedicine providers, cross referenced to geography, specialty, licensing, and practice size or availability.

10. The system of claim 1, wherein each module comprises a software tool that enables communication with one or more distributed network programs.

11. A method for communicating drug information between a patient consumer and a pharmaceutical manufacturer, the method comprising (a) a first user accessing a remote program or database from a first user interface, (b) the first user uploading or downloading user information from said program or database, (c) a second user accessing the remote program or the remote database from a second user interface, and (d) the second user downloading information uploaded by the first user and acting upon said information.

12. The method of claim 11, wherein said remote program or database is accessed by each of the first user interface and the second user interface through a system using distributed computer middleware.

13. The method of claim 11, wherein the database is selected from the group consisting of a database containing diseases and syndromes and their associated symptoms and approved drugs for treatment, a database of de-identified patient consumers cross-referenced to their related symptoms, syndromes, or diseases, a database of approved drugs and drugs currently in clinical trials for specific diseases, symptoms or syndromes, a database of physicians or other healthcare providers and telemedicine providers cross-reference to specialty, licensing, and geography, a database of approved drugs and discounts applied thereto, a database of available clinical trials related to specific drugs and patient participant payments related thereto, a database of currently recruiting clinical trials for particular drugs, indications, subject patient attributes, and geographic area, and a database of healthcare providers, including telemedicine providers, cross referenced to geography, specialty, licensing, and practice size or availability.

14. The method of claim 11, wherein the first user is a patient consumer and the second user is a pharmaceutical manufacturer.

15. The method of claim 11, wherein the first user is a pharmaceutical manufacturer and the second user is a patient consumer.

16. The method of claim 11, wherein the first user is a patient consumer and the second user is a healthcare provider.

17. The method of claim 11, wherein the first user is a healthcare provider and the second user is a patient consumer.

18. The method of claim 11, wherein the first user is a healthcare provider and the second user is a pharmaceutical manufacturer.

19. The method of claim 11, wherein the first user is a pharmaceutical manufacturer and the second user is a healthcare provider.

20. A pharmaceutical drug distribution system comprising:
a distributed network system comprising:
one or more user interfaces,
a central processing unit,
a database of drug and patient information, and
a telecommunications circuit;
configured to deliver patient information to a system administrator;
configured to deliver patient information to a pharmaceutical manufacturer;
configured to deliver drug information to a patient consumer;
configured to deliver patient information to a telemedicine provider;
configured to deliver patient information to a pharmacy; and
configured to deliver patient information to a drug payment medium.
